(19) United States
(12) Patent Application Publication
(10) Pub. No.: US 2022/0401702 A1
(43) Pub. Date: Dec. 22, 2022

(54) Impedance-Determining Medical Systems

(71) Applicant: Bard Access Systems, Inc., Salt Lake City, UT (US)

(72) Inventors: Anthony K. Misener, Bountiful, UT (US); Steffan Sowards, Salt Lake City, UT (US); Robin Scott Urry, Layton, UT (US)

(21) Appl. No.: 17/843,791

(22) Filed: Jun. 17, 2022

Related U.S. Application Data
(60) Provisional application No. 63/212,346, filed on Jun. 18, 2021.

(51) Int. Cl. A61M 25/06; A61M 25/00

(52) U.S. Cl. CPC A61M 25/06; A61M 25/0097; A61M 25/09041

(57) ABSTRACT

Disclosed herein are impedance-determining medical systems. An impedance-determining medical system can include an impedance interrogator and an impedance-sensing medical device. The impedance interrogator can include instructions configured to instantiate one or more processes in random-access memory upon processing by one or more processors that determine impedance from electrical signals corresponding to electrical currents passed through a biological or non-biological material. The impedance-sensing medical device can include two or more longitudinal conductors distributed among one or more pieces of the impedance-sensing medical device and separated by one or more longitudinal insulators. The two-or-more conductors can be configured to emit, detect, or alternately emit and detect via two or more electrodes thereof the electrical currents passed through the biological or non-biological material. The impedance-sensing medical device can be configured to form a direct or indirect connection to the impedance interrogator and provide the electrical signals to the impedance interrogator.

### PRIORITY

This application claims the benefit of priority to U.S. Provisional Patent Application No. 63/212,346, filed Jun. 18, 2021, which is incorporated by reference in its entirety into this application.

### BACKGROUND

Many different medical devices are commonly inserted into the body to diagnose or treat medical conditions. For example, different catheters are used within the body for delivery of fluids including those containing medicaments, removal of bodily fluids, or transport of surgical tools or other instruments. Adding impedance-sensing abilities to medical devices such as catheters, needles, drills, or the like can provide insight into the body including the one or more tissues in which the medical device are used. Disclosed herein are impedance-determining medical systems including impedance interrogators and impedance-sensing medical devices.

### SUMMARY

Disclosed herein is an impedance-sensing medical device including, in some embodiments, a two-piece shaft including an inner tube and an outer tube, two or more longitudinal conductors distributed among the inner tube and the outer tube of the two-piece shaft, and a longitudinal insulator of the inner tube or the outer tube separating the two-or-more longitudinal conductors from each other. The two-or-more conductors are configured to emit, detect, or alternately emit and detect via two or more electrodes thereof electrical currents passed through a biological or non-biological material. In addition, a connector of the impedance-sensing medical device is configured to form a direct or indirect connection to an impedance interrogator for providing electrical signals to the impedance interrogator. The connector includes two or more connection points in correspondence with the two-or-more electrodes.

In some embodiments, the inner tube of the two-piece shaft is formed of the longitudinal insulator or coated with the longitudinal insulator.

In some embodiments, the outer tube of the two-piece shaft is formed of a same or different longitudinal insulator as the inner tube of the two-piece shaft.

In some embodiments, the two-or-more longitudinal conductors are distributed over the inner tube of the two-piece shaft.

In some embodiments, the two-or-more longitudinal conductors are lithographically patterned over the inner tube of the two-piece shaft.

In some embodiments, the inner tube of the two-piece shaft is a needle, and the outer tube of the two-piece shaft is slidably disposed thereover.

In some embodiments, the inner tube of the two-piece shaft is a needle formed of the longitudinal insulator or coated with the longitudinal insulator, the two-or-more longitudinal conductors are distributed by lithographically patterning over the needle, and the outer tube of the two-piece shaft is slidably disposed over the needle. In addition, the outer tube of the two-piece shaft is formed of a same or different longitudinal insulator as the needle.

In some embodiments, the needle is stainless steel coated with the longitudinal insulator. The two-or-more longitudinal conductors are lithographically patterned in a same or different longitudinal insulator over that coating the needle.

Also disclosed herein is an impedance-determining medical system including, in some embodiments, an impedance interrogator and an impedance-sensing medical device. The impedance interrogator includes one or more processors, primary memory including read-only memory (“ROM”) and random-access memory (“RAM”), and instructions stored in the ROM. The instructions are configured to instantiate one or more processes in the RAM for determining impedance from electrical signals corresponding to electrical currents passed through a biological or non-biological material. The impedance-sensing medical device includes two or more longitudinal conductors distributed among one or more pieces of the impedance-sensing medical device and separated by one or more longitudinal insulators. The two-or-more longitudinal conductors are configured to emit, detect, or alternately emit and detect via two or more electrodes thereof the electrical currents passed through the biological or non-biological material. The impedance-sensing medical device is configured to form a direct or indirect connection to the impedance interrogator and provide the electrical signals to the impedance interrogator.

In some embodiments, the impedance-sensing medical device includes the two-or-more longitudinal conductors in a one-piece shaft with two longitudinal conductors separated by two longitudinal insulators.

In some embodiments, each longitudinal conductor of the two longitudinal conductors forms less than half the shaft. Each longitudinal insulator of the two longitudinal insulators is disposed between the two longitudinal conductors on an opposite side of the shaft from another longitudinal insulator of the two longitudinal insulators.

In some embodiments, the shaft has a symmetry such that a longitudinal plane of symmetry passes through either the two longitudinal conductors or the two longitudinal insulators but not both the two longitudinal conductors and the two longitudinal insulators.

In some embodiments, the impedance-sensing medical device is a needle configured for at least vascular access.

In some embodiments, the needle includes a needle hub over a proximal portion of the shaft and a needle tip formed in a distal portion of the shaft.

In some embodiments, the impedance-determining medical system further includes a needle guide. The needle guide is configured to couple with a needle-guide attachment point of an ultrasound probe as the impedance interrogator or a portion of the impedance interrogator. The needle guide, the needle-guide attachment point, and the ultrasound probe include electrical circuitry configured to operably connect the needle to the ultrasound probe when a) the needle guide is coupled with the needle-guide attachment point and b) the needle is inserted into an aperture of the needle guide. When the needle is inserted into the aperture of the needle guide the two longitudinal conductors make electrical connections with two opposing electrical contacts within the aperture.

In some embodiments, the needle guide includes conductive inward-facing protrusions configured to establish an electrical connection within outward-facing receptacles of the needle-guide attachment point when the needle guide is coupled with the needle-guide attachment point.

In some embodiments, the protrusions include barrier-piercing contact points configured to pierce a protective film-based barrier when used over the ultrasound probe. The barrier-piercing contact points are also configured to establish the electrical connection with contact points within the receptacles of the needle-guide attachment point. The receptacles are shaped to accommodate the barrier-piercing contact points of the protrusions.

In some embodiments, the impedance-determining medical system further includes an alligator-clamp connecting device. The alligator-clamp connecting device includes a proximal connecting-device connector, a distal connecting-device connector, and electrical circuitry therebetween. The proximal connecting-device connector is configured to couple with an impedance-interrogator connector of the impedance interrogator. The distal connecting-device connector includes an alligator clamp configured to couple with the impedance-sensing medical device.

In some embodiments, the alligator clamp includes a pair of jaws. Each jaw of the pair of jaws includes a plurality of teeth configured to establish an electrical connection with a longitudinal conductor of the two longitudinal conductors when the alligator clamp is clamped over the shaft.

In some embodiments, the teeth include barrier-piercing contact points configured to pierce a protective film-based barrier when used over a portion of the shaft.

In some embodiments, the impedance-sensing medical device includes the two-or-more longitudinal conductors in a one-piece shaft with two longitudinal conductors separated by one longitudinal insulator.

In some embodiments, each longitudinal conductor of the two longitudinal conductors is primarily disposed on an opposite side of the shaft from another longitudinal conductor of the two longitudinal conductors. Sides of the shaft are a luminal side of the shaft and an abluminal side of the shaft.

In some embodiments, the two longitudinal conductors respectively include two contact points for establishing electrical connections. The two contact points are on the abluminal side of the shaft.

In some embodiments, the two longitudinal conductors respectively include two contact points for establishing electrical connections. The two contact points are apportioned between the luminal side of the shaft and an abluminal side of the shaft.

In some embodiments, the impedance-sensing medical device is a needle configured for at least intraosseous access.

In some embodiments, the impedance interrogator is an intraosseous drill. The intraosseous drill includes a needle hub configured to hold a proximal portion of the shaft and operably connect the needle to the intraosseous drill. The needle is operably connected to the intraosseous drill when the needle is inserted into the needle hub and the two longitudinal conductors establish the electrical connections with two electrical contacts within the needle hub.

In some embodiments, the intraosseous drill includes an obturator hub over the needle hub. The obturator hub is configured to hold a proximal portion of an insulated obturator or an obturator of an insulator material.

In some embodiments, the impedance-sensing medical device includes the two-or-more longitudinal conductors in a two-piece shaft with two longitudinal conductors separated by one longitudinal insulator.

In some embodiments, the impedance-sensing medical device includes a combination of a needle and an obturator disposed in the needle configured for at least intraosseous access.

In some embodiments, the longitudinal insulator is disposed over a luminal side of the needle or an abluminal side of the obturator.

In some embodiments, the impedance interrogator is an intraosseous drill. The intraosseous drill includes a needle hub configured to hold a proximal portion of the needle and operably connect the needle to the intraosseous drill. The needle is operably connected to the intraosseous drill when the needle is inserted into the needle hub and a longitudinal conductor of the needle establishes an electrical connection with an electrical contact within the needle hub.

In some embodiments, the intraosseous drill includes an obturator hub over the needle hub. The obturator hub is configured to hold a proximal portion of the obturator and operably connect the obturator to the intraosseous drill. The obturator is operably connected to the intraosseous drill when the obturator is inserted into the obturator hub and a longitudinal conductor of the obturator establishes an electrical connection with an electrical contact within the obturator hub.

In some embodiments, the impedance-sensing medical device includes the two-or-more longitudinal conductors distributed among two pieces of the impedance-sensing medical device. The two pieces of the impedance-sensing medical device include a catheter and a needle disposed in the catheter configured for at least vascular access.

In some embodiments, the two-or-more longitudinal conductors include a needle shaft of the needle and one or more conductive filaments disposed in a catheter wall of a catheter tube of the catheter. The needle shaft and the one-or-more conductive filaments are separated by the catheter wall as the insulator of the one-or-more insulators.

In some embodiments, the impedance-sensing medical device includes the two-or-more longitudinal conductors distributed in one piece of the impedance-sensing medical device. The one piece of the impedance-sensing medical device includes a catheter configured for at least vascular access.

In some embodiments, the two-or-more longitudinal conductors include two-or-more conductive filaments disposed in a catheter wall of the catheter tube of the catheter. The two-or-more conductive filaments are separated by the catheter wall as the insulator of the one-or-more insulators.

In some embodiments, the impedance-sensing medical device includes the two-or-more longitudinal conductors distributed in one piece of the impedance-sensing medical device. The one piece of the impedance-sensing medical device includes a guidewire configured for at least vascular access or vascular guidance.

In some embodiments, the two-or-more longitudinal conductors include a twisted pair of conductive filaments. At least one conductive filament of the conductive filaments is coated with a polymeric material as the insulator of the one-or-more insulators.

In some embodiments, the two-or-more longitudinal conductors include a wound pair of conductive filaments with a wound conductive filament wound around a core conductive filament. At least one conductive filament of the conductive filaments is coated with a polymeric material as the insulator of the one-or-more insulators.

These and other features of the concepts provided herein will become more apparent to those of skill in the art in view of the accompanying drawings and following description, which describe particular embodiments of such concepts in greater detail.

### DRAWINGS

FIG. 1 illustrates an impedance-determining medical system including an impedance interrogator and an impedance-sensing medical device in relation to a patient in accordance with some embodiments.

FIG. 2 illustrates longitudinal conductors of a portion of an impedance-sensing medical device in accordance with some embodiments.

FIG. 3 illustrates a cross section of the portion of the impedance-sensing medical device of FIG. 2 in accordance with some embodiments.

FIG. 4 illustrates a first view of a distal portion of the impedance-sensing medical device of FIGS. 2 and 3 configured as a needle in accordance with some embodiments.

FIG. 5 illustrates a second view of the needle in accordance with some embodiments.

FIG. 6 illustrates a third view of the needle in accordance with some embodiments.

FIG. 7 illustrates a first view of a needle guide as a connecting device for connecting the needle to an ultrasound probe in accordance with some embodiments.

FIG. 8 illustrates a second view of the needle guide for connecting the needle to the ultrasound probe in accordance with some embodiments.

FIG. 9 illustrates a distal connecting-device connector of an alligator-clamp connecting device as the connecting device for connecting the needle to the impedance interrogator in accordance with some embodiments.

FIG. 10 illustrates the distal connecting-device connector of the alligator-clamp connecting device connected to the needle of the impedance-sensing medical device in accordance with some embodiments.

FIG. 11 illustrates longitudinal conductors of a portion of an impedance-sensing medical device in accordance with some embodiments.

FIG. 12A illustrates a first cross section of the portion of the impedance-sensing medical device of FIG. 11 in accordance with some embodiments.

FIG. 12B illustrates a second cross section of the portion of the impedance-sensing medical device of FIG. 11 in accordance with some embodiments.

FIG. 13 illustrates the impedance-sensing medical device of FIG. 11, 12A, or 12B configured as an intraosseous needle disposed in an intraosseous drill as the impedance interrogator in accordance with some embodiments.

FIG. 14 illustrates a cross section of a needle hub as a connecting device for connecting the intraosseous needle to the intraosseous drill in accordance with some embodiments.

FIG. 15 illustrates inserting a proximal portion of the intraosseous needle into the needle hub in accordance with some embodiments.

FIG. 16 illustrates the proximal portion of the intraosseous needle within the needle hub in accordance with some embodiments.

FIG. 17 illustrates a cross section of the proximal portion of the intraosseous needle within the needle hub in accordance with some embodiments.

FIG. 18 illustrates inserting the needle hub into the proximal portion of the intraosseous needle in accordance with some embodiments.

FIG. 19 illustrates the needle hub within the proximal portion of the intraosseous needle in accordance with some embodiments.

FIG. 20 illustrates a cross section of the needle hub within the proximal portion of the intraosseous needle in accordance with some embodiments.

FIG. 21 illustrates the proximal portion of the intraosseous needle within the needle hub in accordance with some embodiments.

FIG. 22 illustrates longitudinal conductors of a portion of an impedance-sensing medical device in accordance with some embodiments.

FIG. 23 illustrates a cross section of the portion of the impedance-sensing medical device of FIG. 22 in accordance with some embodiments.

FIG. 24 illustrates the impedance-sensing medical device of FIGS. 22 and 23 configured as a catheter and a needle disposed in the catheter in accordance with some embodiments.

FIG. 25 illustrates longitudinal conductors of a portion of an impedance-sensing medical device in accordance with some embodiments.

FIG. 26 illustrates a cross section of the portion of the impedance-sensing medical device of FIG. 25 in accordance with some embodiments.

FIG. 27 illustrates the impedance-sensing medical device of FIGS. 25 and 26 configured as a catheter in accordance with some embodiments.

FIG. 28 illustrates an impedance-sensing medical device configured as a guidewire in accordance with some embodiments.

FIG. 29 illustrates the guidewire in accordance with some embodiments.

FIG. 30 illustrates the guidewire in accordance with some other embodiments.

FIG. 31 illustrates an impedance-sensing medical device configured as a needle with an outer tube slidably disposed thereover in accordance with some embodiments.

FIG. 32 illustrates a cross section of a portion of the impedance-sensing medical device of FIG. 31 in accordance with some embodiments.

FIG. 33 illustrates a block diagram of an ultrasound system in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

As set forth above, many different medical devices are commonly inserted into the body to diagnose or treat medical conditions. For example, different catheters are used within the body for delivery of fluids including those containing medicaments, removal of bodily fluids, or transport of surgical tools or other instruments. Adding impedance-sensing abilities to medical devices such as catheters, needles, drills, or the like can provide insight into the body including the one or more tissues in which the medical device are used. Disclosed herein are impedance-determining medical systems including impedance interrogators and impedance-sensing medical devices.

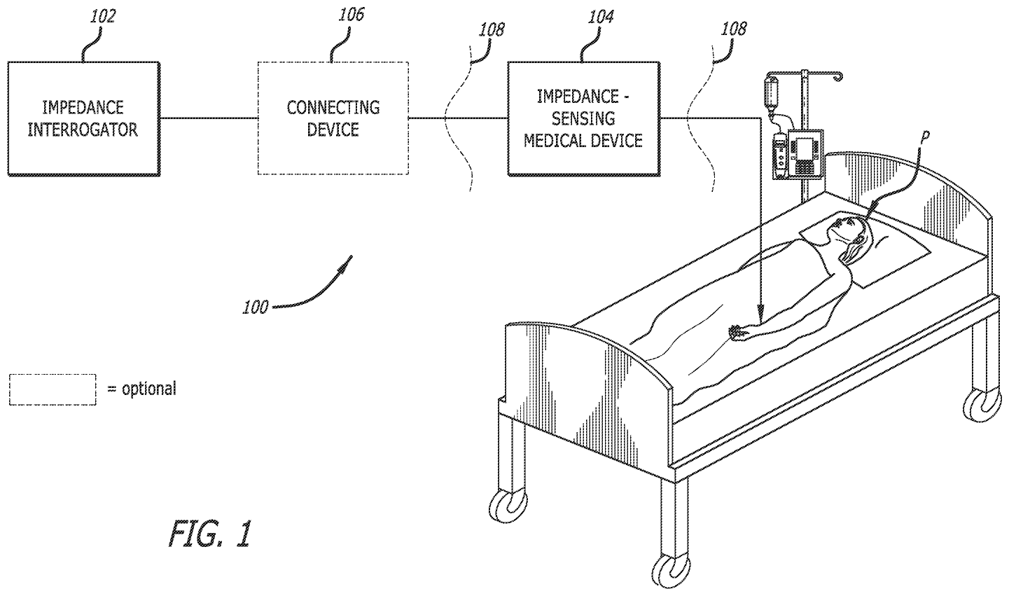

FIG. 1 illustrates an impedance-determining medical system 100 in relation to a patient P in accordance with some embodiments.

As shown, the impedance-determining medical system 100 includes an impedance interrogator 102, an impedance-sensing medical device 104, and an optional connecting device 106 between the impedance interrogator 102 and the impedance-sensing medical device 104, each of which is set forth in more detail below by way of examples thereof. Notably, the connecting device 106, when present, can be configured for breaching a procedural barrier 108 such as a sterile drape defining a procedural field such as a sterile field. For example, the needle guide 122 and the alligator-clamp connecting device 178 are two connecting devices set forth below for breaching the procedural barrier 108. Alternatively, the impedance-sensing medical device 104, itself, can be configured to breach the procedural barrier 108. For example, the needle 116, the intraosseous needle 188, the catheter 208 or 220, the guidewire 230, etc. can be configured to breach the procedural barrier 108 or at least pass therethrough into the patient P.

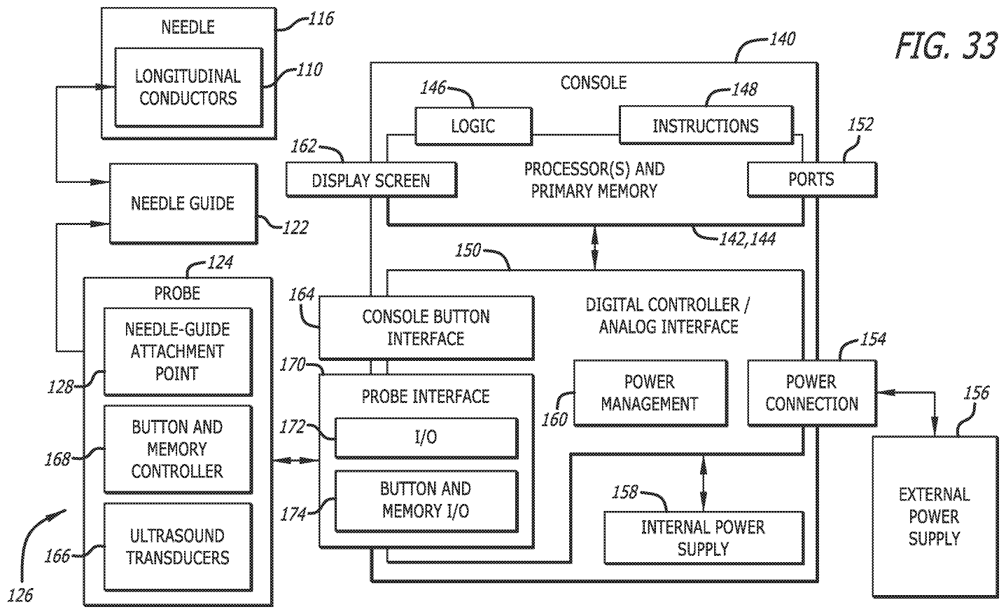

The impedance interrogator 102 can include a single impedance-interrogating device such as the intraosseous drill 190 up to a system including impedance interrogation as one or more of its functions such as the ultrasound system 126, which includes the ultrasound probe 124 and the console 140 operating together. In any case, the impedance interrogator 102 includes one or more processors, primary memory including ROM and RAM, and instructions stored in the ROM. The instructions are configured to instantiate one or more processes in the RAM for determining impedance from electrical signals corresponding to electrical currents passed through biological materials such as one or more tissues of the patient P or non-biological materials such as saline, contrast media, or the like and, subsequently, identifying the biological of non-biological material. (See, for example, FIG. 33, which includes a block diagram of the ultrasound system 126 including the foregoing electronic components and instructions.)

The impedance-sensing medical device 104 includes two or more longitudinal conductors 110 and one or more longitudinal insulators 112. The two-or-more longitudinal conductors 110 are distributed among one or more pieces of the impedance-sensing medical device 104 and separated by the one-or-more longitudinal insulators 112. The two-or-more longitudinal conductors 110 are configured to emit, detect, or alternately emit and detect via two or more electrodes (e.g., terminal portions of any of the two-or-more longitudinal conductors 110 disclosed herein) electrical currents passed through the biological materials such as the one-or-more tissues of the patient P or the non-biological materials such as the saline, contrast media, or the like. The impedance-sensing medical device 104 is configured to form a direct connection to the impedance interrogator 102 or indirect connection to the impedance interrogator 102 via the connecting device 106 such that the impedance-sensing medical device 104 can provide the electrical signals to the impedance interrogator 102.

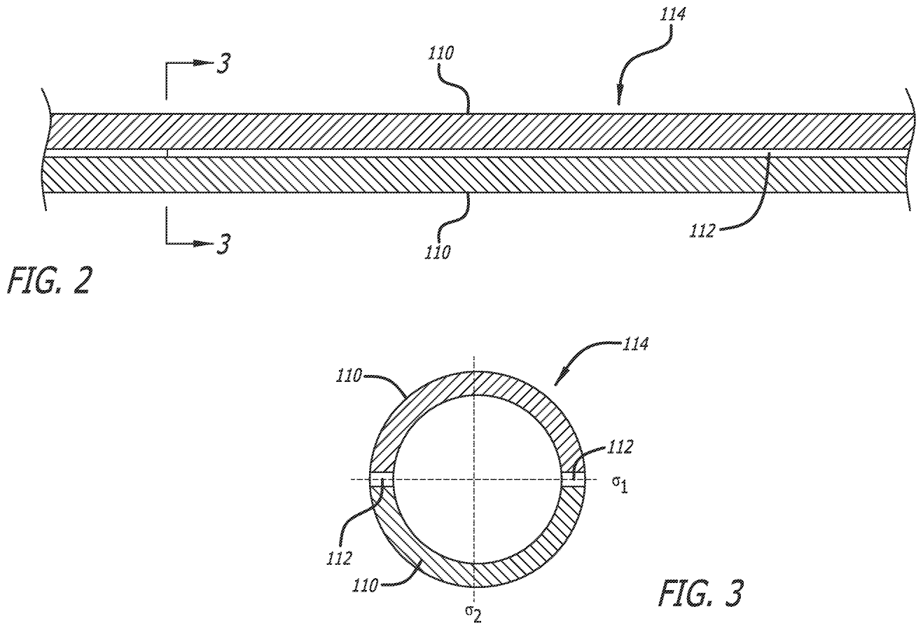

FIG. 2 illustrates the two-or-more longitudinal conductors 110 of a portion of the impedance-sensing medical device 104 in accordance with some embodiments. FIG. 3 illustrates a cross section of the portion of the impedance-sensing medical device 104 of FIG. 2 in accordance with some embodiments.

As shown, the impedance-sensing medical device 104 can include the two-or-more longitudinal conductors 110 in a one-piece shaft 114 with two longitudinal conductors 110 separated by two longitudinal insulators 112. Each longitudinal conductor of the two longitudinal conductors 110 forms less than half (e.g., a sagittal or coronal half) of the shaft 114. Indeed, each longitudinal insulator of the two longitudinal insulators 112 is disposed between the two longitudinal conductors 110 on an opposite side of the shaft 114 from another longitudinal insulator of the two longitudinal insulators 112. Such a configuration of the shaft 114 has a symmetry such that a longitudinal plane of symmetry (e.g., σ₁ or σ₂ in FIG. 3) passes through either the two longitudinal conductors 110 or the two longitudinal insulators 112 but not both the two longitudinal conductors 110 and the two longitudinal insulators 112.

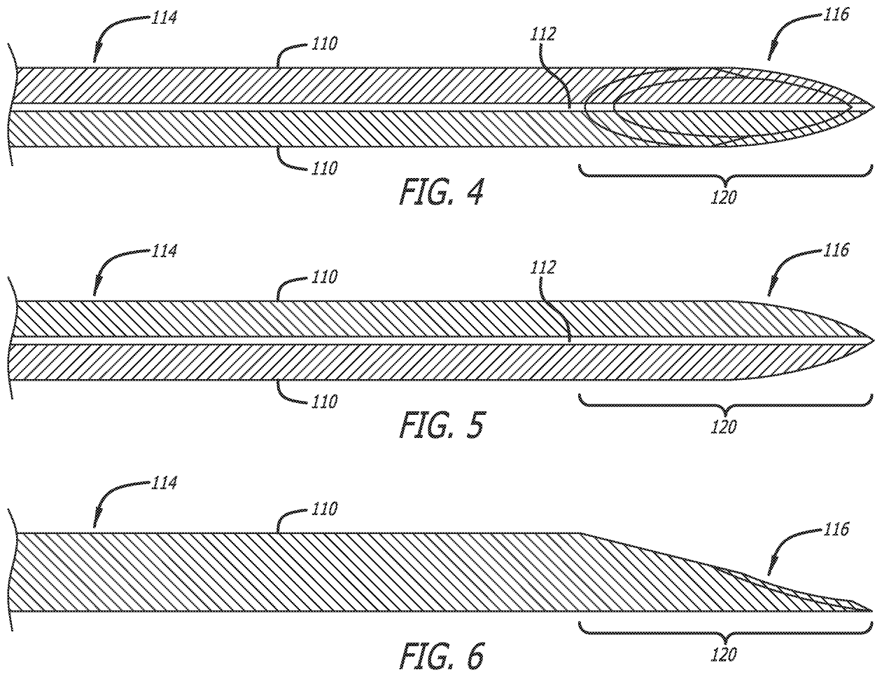

Notwithstanding the foregoing, the shaft 114 can include an additional longitudinal insulator 112 over a luminal or abluminal surface of the shaft 114 or two additional insulators 112 over both the luminal and abluminal surfaces of the shaft 114 provided an entirety of the shaft 114 is not covered, particularly surface portions of the shaft 114 configured for making electrical connections. In an example, the shaft 114 can include the additional longitudinal insulator 112 over the luminal surface of the shaft 114 such that the additional longitudinal insulator is over the luminal surface of the shaft 114 shown in FIG. 3. In another example, the shaft 114 can include the additional longitudinal insulator 112 over the abluminal surface of the shaft 114 such that the additional longitudinal insulator is over the abluminal surface of the shaft 114 shown in FIG. 4.

FIGS. 4-6 illustrate different views of a distal portion of the impedance-sensing medical device 104 of FIGS. 2 and 3 configured as a needle 116 in accordance with some embodiments.

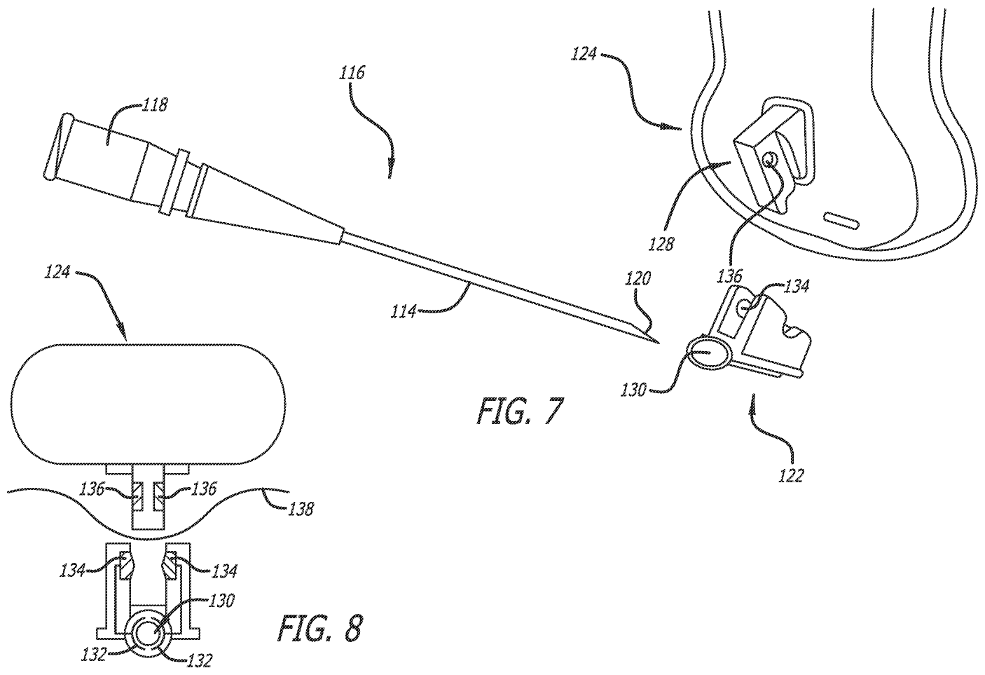

The impedance-sensing medical device 104 including the foregoing shaft 114 can be the needle 116, which can be configured for at least vascular access. The needle 116 includes a needle hub 118 over a proximal portion of the shaft 114 (see FIG. 7) and a needle tip 120 (e.g., a beveled needle tip) formed in a distal portion of the shaft 114 as shown in FIGS. 4-6.

Notably, the impedance-sensing medical device 104 including the shaft 114 can alternatively be a cannula, stylet, or the like in some embodiments.

FIGS. 7 and 8 illustrate different views of a needle guide 122 as the connecting device 106 for connecting the needle 116 to an ultrasound probe 124 of an ultrasound system 126 in accordance with some embodiments.

As shown, the needle guide 122 is configured to couple with a needle-guide attachment point 128 of the ultrasound probe 124 as the impedance interrogator 102 or at least a portion of the impedance interrogator 102 (e.g., the impedance interrogator 102 can be the ultrasound system 126, of which the ultrasound probe 124 is a portion). The needle guide 122, the needle-guide attachment point 128, and the ultrasound probe 124 include electrical circuitry or electronic circuitry with any needed electronic components configured to operably connect the needle 116 to the ultrasound probe 124 when both the needle guide 122 is coupled with the needle-guide attachment point 128 and the needle 116 is inserted into an aperture 130 of the needle guide 122. When the needle 116 is inserted into the aperture 130 of the needle guide 122 the two longitudinal conductors 110 make electrical connections with two opposing electrical contacts 132 within the aperture 130.

Notably, the needle guide 122 includes conductive, inward-facing protrusions 134 configured to establish an electrical connection within outward-facing receptacles 136 of the needle-guide attachment point 128 when the needle guide 122 is coupled with the needle-guide attachment point 128. As shown, the protrusions 134 include barrier-piercing contact points configured to pierce a protective film-based barrier or probe cover 138 when used over the ultrasound probe 124. The barrier-piercing contact points are also configured to establish the electrical connection with contact points within the receptacles 136 of the needle-guide attachment point 128. The receptacles 136 are shaped to accommodate the barrier-piercing contact points of the protrusions 134.

FIG. 33 illustrates a block diagram of the ultrasound system 126 in accordance with some embodiments.

Adverting to FIG. 33 to describe certain aspects of the ultrasound system 126 as the impedance interrogator 102, the ultrasound system 126 includes the ultrasound probe 124 and a console 140 that houses a variety of components of the ultrasound system 126. Like that set forth above, the console 140 includes one or more processors 142 and primary memory 144 such as ROM (e.g., electrically erasable programmable read-only memory [“EEPROM”]) and RAM is included in the console 140 for controlling various functions of the ultrasound system 126, as well as executing various logic operations of logic 146 during operation of the ultrasound system 126. As for operating the ultrasound system 126, the console 140 is configured to instantiate by way of instructions 148 stored in the ROM one or more console processes in the RAM for ultrasound imaging and impedance interrogation with the ultrasound probe 124 and the needle 116 when operably connected to the console 140. A digital controller/analog interface 150 is included with the console 140 and is in communication with the one-or-more processors 142 and other system components to govern interfacing between the ultrasound probe 124 and the other system components.

The console 140 further includes ports 152 for connection with additional components. The ports 152 can be universal serial bus (“USB”) ports, though other types of ports can be used for this connection or any other connections shown or described herein. A power connection 154 is included with the console 140 to enable operable connection to an external power supply 156. An internal power supply 158 (e.g., a battery) can also be employed either with or exclusive of the external power supply 156. Power management circuitry 160 is included with the digital controller/analog interface 150 of the console 140 to regulate power use and distribution.

A display screen 162 (e.g., a liquid-crystal display [“LCD”] screen) is operably connected to the console 140. As shown, the display screen 162 can be integrated into the console 140 to provide a graphical user interface (“GUI”) and display information for a clinician during ultrasound imaging or impedance interrogation. Alternatively, the display screen 162 is separate from the console 140 and communicatively coupled thereto. A console button interface 164 and control buttons included on the ultrasound probe 124 can be used to immediately call up a desired mode to the display screen 162 by the clinician for the ultrasound imaging, impedance interrogation, or both.

The ultrasound probe 124 includes a probe head that houses an array of ultrasound transducers 166, wherein the ultrasound transducers 166 are piezoelectric transducers or capacitive micromachined ultrasound transducers (“CMUTs”). The ultrasound transducers 166 are configured to emit ultrasound pulses into at least the one-or-more tissues of the patient P and receive reflected ultrasound pulses reflected back through the one-or-more tissues for producing ultrasound images. The probe head is configured for placement against skin of the patient P. In this way, the ultrasound system 126, by way of the ultrasound probe 124 and the logic 146, can provide the ultrasound imaging.

The ultrasound probe 124 also includes a button-and-memory controller 168 for governing button operation, as well as governing operation of the ultrasound probe 124. The button-and-memory controller 168 can include ROM (e.g., EEPROM). The button-and-memory controller 168 is in operable communication with a probe interface 170 of the console 140, which includes an input/output (“I/O”) component 172 for interfacing with the ultrasound transducers 166 and a button-and-memory I/O component 174 for interfacing with the button-and-memory controller 168.

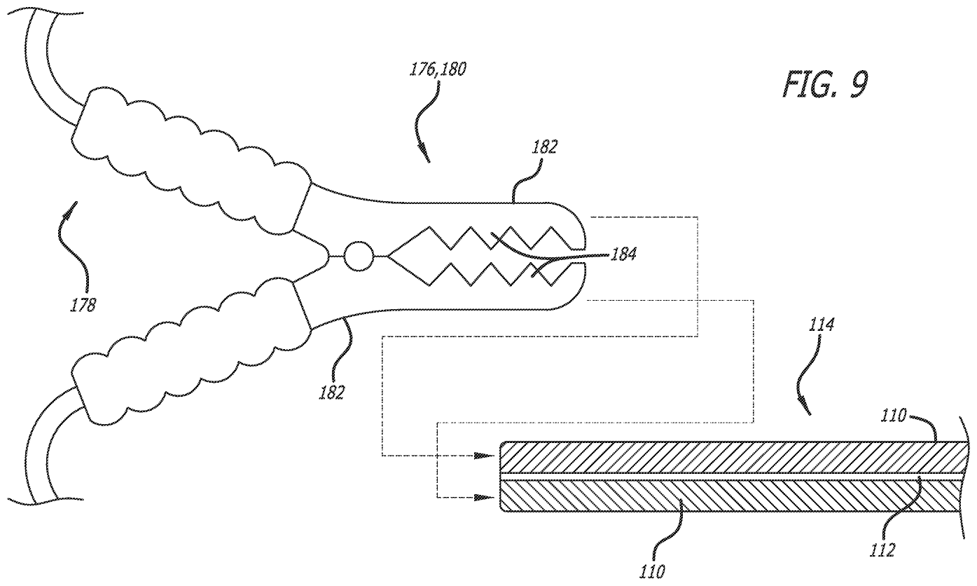

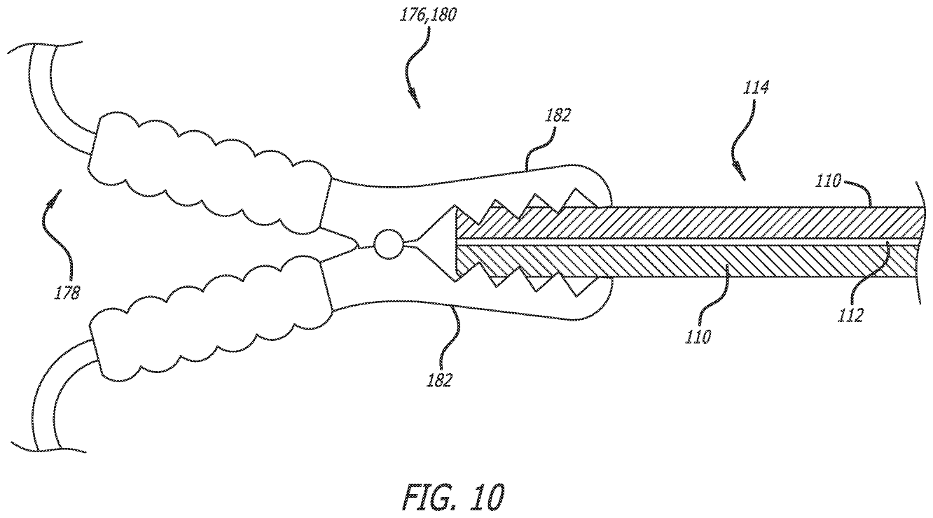

FIG. 9 illustrates a distal connecting-device connector 176 of an alligator-clamp connecting device 178 as the connecting device 106 for connecting the needle 116 to the impedance interrogator 102 in accordance with some embodiments. FIG. 10 illustrates the distal connecting-device connector 176 of the alligator-clamp connecting device 178 connected to the needle 116 of the impedance-sensing medical device 104 in accordance with some embodiments.

The alligator-clamp connecting device 178 includes a proximal connecting-device connector, the distal connecting-device connector 176, and electrical circuitry or electronic circuitry with any needed electronic components between the proximal connecting-device connector and the distal connecting-device connector 176. While not shown, the proximal connecting-device connector is configured to couple with an impedance-interrogator connector of the impedance interrogator 102. The distal connecting-device connector 176 includes an alligator clamp 180 configured to couple with the needle 116, a cannula, a stylet, or the like of the impedance-sensing medical device 104 as shown in FIG. 10.

The alligator clamp 180 includes a pair of 182. Each jaw of the pair of 182 includes a plurality of teeth 184 configured to establish an electrical connection with a longitudinal conductor of the two longitudinal conductors 110 when the alligator clamp 180 is clamped over the shaft 114 such as the proximal portion the shaft 114 including a proximal end of the shaft 114, as shown, or the proximal portion of the shaft 114 distal of the needle hub 118, if present. Notably, the teeth 184 can include barrier-piercing contact points configured to pierce a protective film-based barrier when used over the proximal portion of the shaft 114.

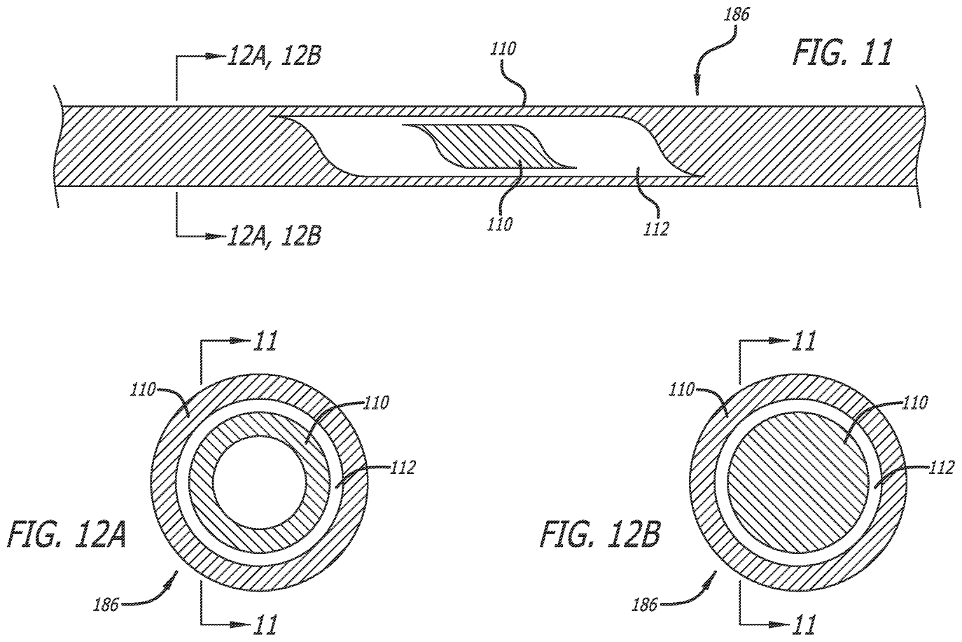

FIG. 11 illustrates the two-or-more longitudinal conductors 110 of a portion of the impedance-sensing medical device 104 in accordance with some embodiments. FIGS. 12A and 12B illustrate different cross sections of the portion of the impedance-sensing medical device 104 of FIG. 11 in accordance with some embodiments.

As shown, the impedance-sensing medical device 104 can include the two-or-more longitudinal conductors 110 in a one- or two-piece shaft 186 with two longitudinal conductors 110 separated by one longitudinal insulator 112.

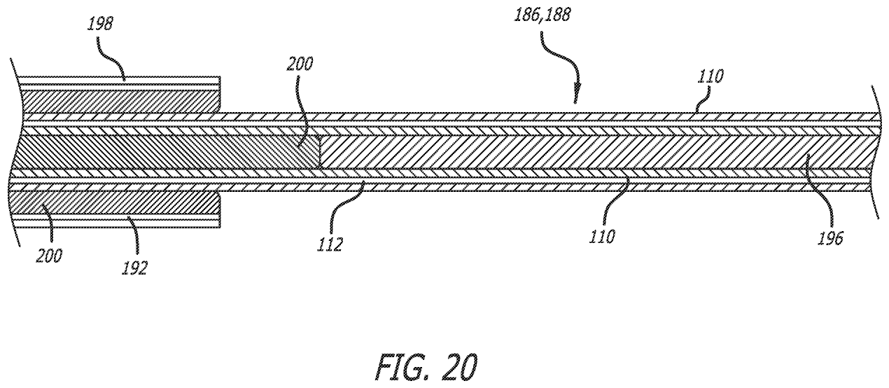

As to the one-piece shaft 186, each longitudinal conductor of the two longitudinal conductors 110 is primarily disposed on an opposite side of the shaft 186 from another longitudinal conductor of the two longitudinal conductors 110, wherein sides of the shaft 186 are a luminal side of the shaft 186 and an abluminal side of the shaft 186. Such a configuration of the shaft 186 has, in effect, an inner tube and an outer tube of the two longitudinal conductors 110 separated by an intervening tube of the longitudinal insulator 112 in at least some embodiments. (See, for example, FIGS. 12A, 17, and 20.) The inner tube of the two longitudinal conductors 110, the intervening tube of the longitudinal insulator 112, and the outer tube of the two longitudinal conductors 110 can be fitted together in successive engineering fits. Alternatively, or in some combination with the foregoing, the intervening tube of the longitudinal insulator 112 can be formed over the inner tube of the two longitudinal conductors 110 by extruding the intervening tube of the longitudinal insulator 112 over the inner tube of the two longitudinal conductors 110 or even coating (e.g., dip coating) the intervening tube of the longitudinal insulator 112 over the inner tube of the two longitudinal conductors 110. The outer tube of the two longitudinal conductors 110 can be formed over the longitudinal insulator 112 by coating (e.g., chemical vapor deposition, physical vapor deposition, plating, etc.) the outer tube of the two longitudinal conductors 110 over the intervening tube of the longitudinal insulator 112. However, the inner tube and the outer tube need not remain in such a configuration over an entirety of the shaft 186 like that shown in FIG. 20. Indeed, FIG. 17 illustrates the inner tube commensurate with the outer tube in the proximal portion of the shaft 186 including the proximal end.

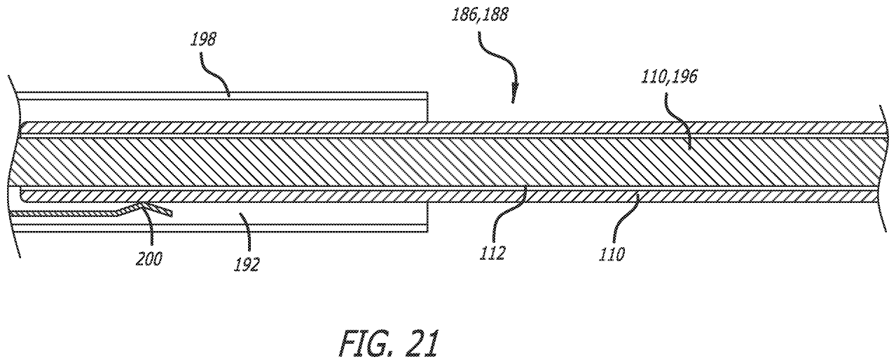

As to the two-piece shaft 186, each longitudinal conductor of the two longitudinal conductors 110 is present in a separate piece of the shaft 186. The two longitudinal conductors 110 can be configured as set forth above for the one-piece shaft 186, albeit separable to expose, for example, a luminal surface of the outer tube of the shaft 186 or an abluminal surface of the inner tube of the shaft 186 when separated. As an alternative to the foregoing inner tube disposed in the outer tube of the shaft 186, however, the two longitudinal conductors 110 can include an inner rod of the shaft 186 disposed in the outer tube of the shaft 186 separated by an intervening tube of the longitudinal insulator 112 as shown in FIGS. 12B and 21. The luminal side of the outer tube or outside of the inner rod can include the intervening tube of the longitudinal insulator 112 disposed thereover. Indeed, as best shown in FIG. 21, the intervening tube of the longitudinal insulator 112 is disposed over the luminal side of the outer tube. That said, the outside of the inner rod can include the intervening tube of the longitudinal insulator 112 disposed thereover to form the impedance-sensing medical device 104 having the outer tube of the shaft 186 and an insulated inner rod as the two longitudinal conductors 110. Notably, the outer tube of the shaft 186, the inner rod of the shaft 186, or both can be lithographically patterned with insulating and conducting materials such that the outer tube of the shaft 186 or the inner rod of the shaft 186 include a different distribution of the two longitudinal conductors, more than the two longitudinal conductors 110, or a combination thereof, optionally, with electrical communication between two or more of the longitudinal conductors 110 of the outer tube of the shaft 186, the inner rod of the shaft 186. As an alternative to the outer tube of the shaft 186 and the insulated inner rod including the two longitudinal conductors 110, the outer tube of the shaft 186 can include its longitudinal conductor 110 and the insulated inner rod can include two longitudinal conductors 110. Despite its longitudinal conduct 110, the outer tube of the shaft 186 need not function as such when the inner rod includes the two longitudinal conductors 110.

Notwithstanding the foregoing, the one- or two-piece shaft 186 can include an additional longitudinal insulator 112 over a luminal or abluminal (or outside) surface of the shaft 186 or two additional insulators 112 over both the luminal and abluminal surfaces of the shaft 186 provided an entirety of the shaft 186 is not covered, particularly surface portions of the shaft 186 configured for making electrical connections. In an example, the one- or two-piece shaft 186 can include the additional longitudinal insulator 112 over the luminal surface of the shaft 186 such that the additional longitudinal insulator is over the luminal surface of the inner tube of the shaft 186 shown in FIG. 12A. In another example, the one- or two-piece shaft 186 can include the additional longitudinal insulator 112 over the abluminal surface of the shaft 186 such that the additional longitudinal insulator is over the abluminal surface of the outer tube of the shaft 186 shown in FIG. 12A. In another example, the one- or two-piece shaft 186 can include the additional longitudinal insulator 112 over the outside surface of the shaft 186 such that the additional longitudinal insulator is over the outside surface of the outer tube of the shaft 186 shown in FIG. 12B.

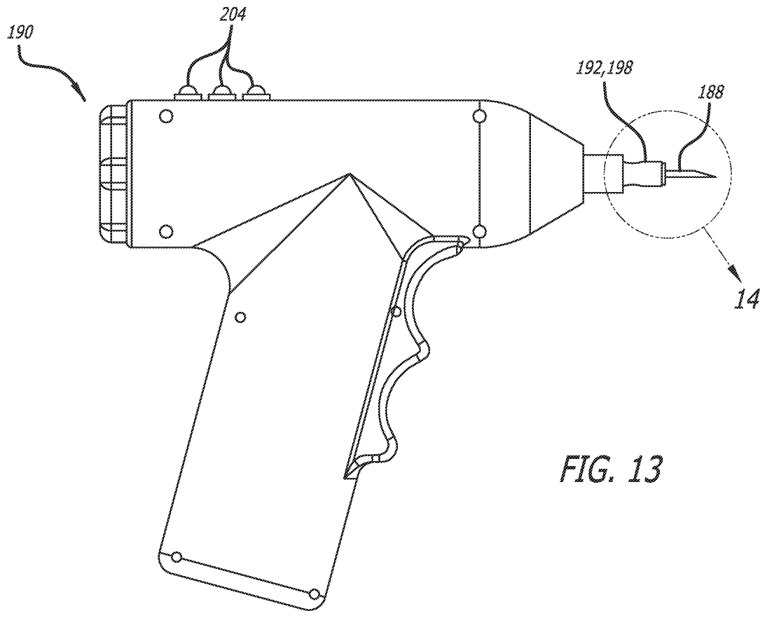

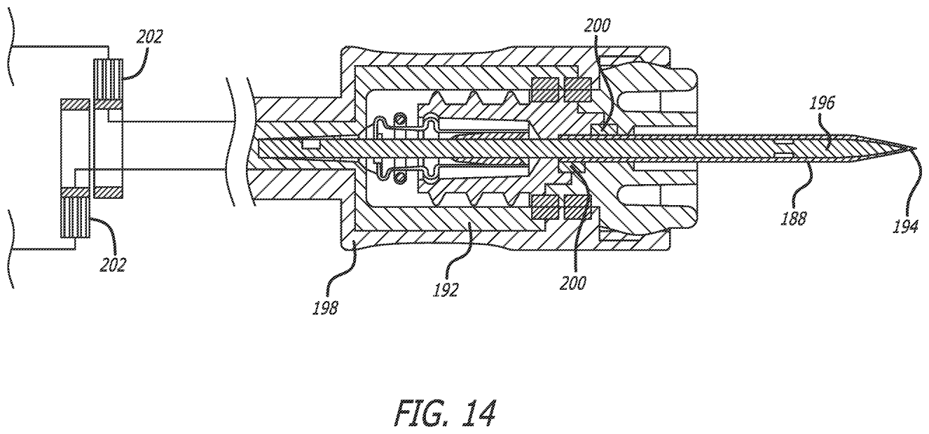

FIG. 13 illustrates the impedance-sensing medical device 104 configured as an intraosseous needle 188 disposed in an intraosseous drill 190 as the impedance interrogator 102 in accordance with some embodiments. FIG. 14 illustrates a cross section of a needle hub 192 as the connecting device 106 for connecting the intraosseous needle 188 to the intraosseous drill 190 in accordance with some embodiments. However, it should be understood that the foregoing embodiments are non-limiting examples. Indeed, the impedance interrogator 102 can be a drill configured for any type of tissue of tissues, and the impedance-sensing medical device 104 can likewise be a corresponding needle for the foregoing tissue or tissues.

The impedance-sensing medical device 104 including at least a portion of the foregoing shaft 186 (e.g., the outer tube) can be the intraosseous needle 188, which can be configured for at least intraosseous access. The intraosseous needle 188 includes a proximal portion of the shaft 186 without a fixedly coupled needle hub like that of the needle 116 of FIG. 7 and a distal portion of the shaft 186 including a needle tip 194 as shown in FIG. 14. As set forth in more detail below, the impedance-sensing medical device can further include an obturator 196 disposed in the intraosseous needle 188, the obturator 196 forming at least another portion of the foregoing shaft 186 (e.g., the inner rod), thereby forming a combination of the intraosseous needle 188 and the obturator 196 disposed in the intraosseous needle 188 configured for at least intraosseous access.

Notably, the impedance-sensing medical device 104 including the foregoing shaft 186 or any of the portions thereof can alternatively be a cannula, stylet, or the like in some embodiments.

The intraosseous drill 190 includes the needle hub 192 as well as an obturator hub over the needle hub 192. The needle hub 192 is configured to hold the proximal portion of the shaft and operably connect the intraosseous needle 188 to the intraosseous drill 190. The obturator hub 198 is configured to hold a proximal portion of the obturator 196 and operably connect the obturator 196 to the intraosseous drill 190. The obturator 196 is insulated from the intraosseous needle 188 by way of the longitudinal insulator 112 disposed over a luminal surface of the intraosseous needle 188, the longitudinal insulator 112 disposed over an outside surface of the obturator 196, or the obturator 196 being of an insulator material.

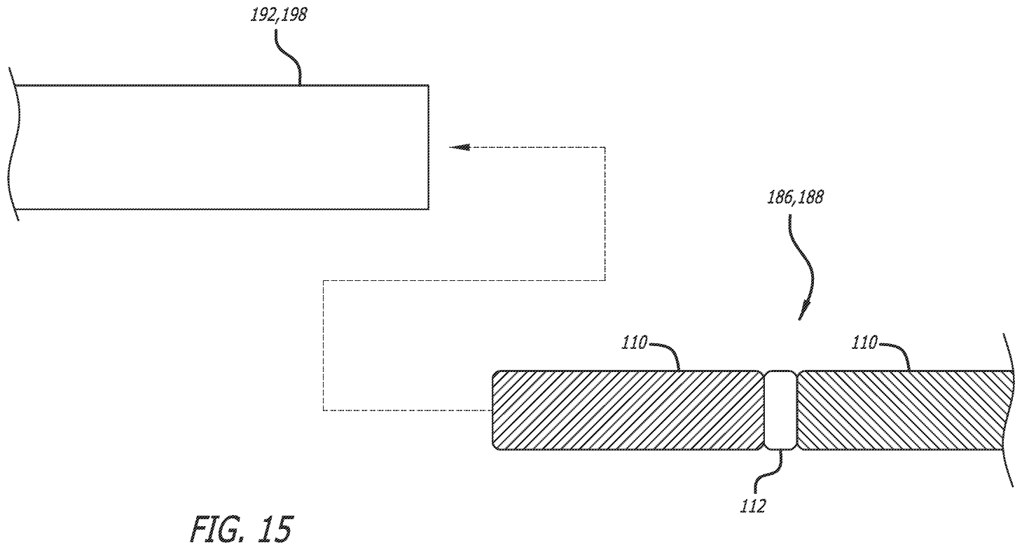

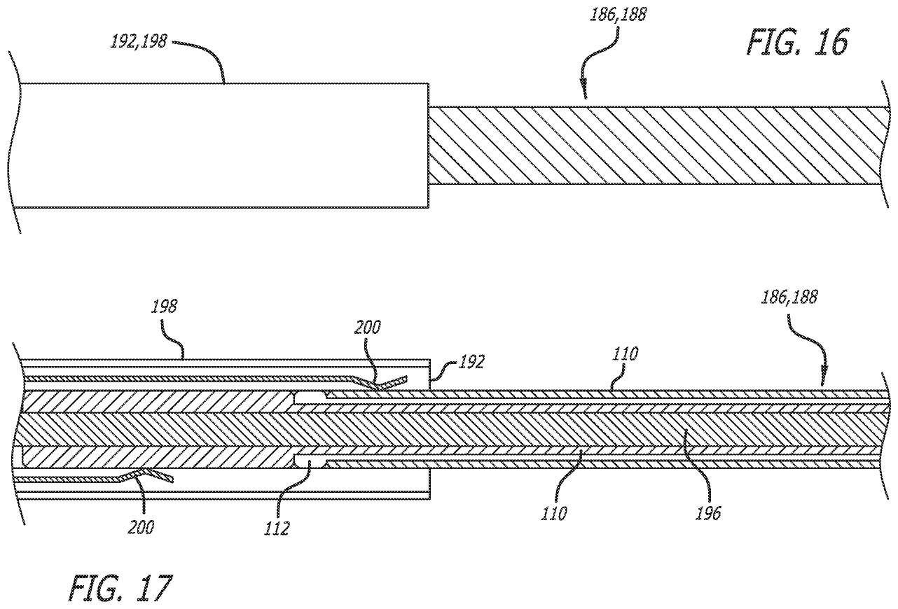

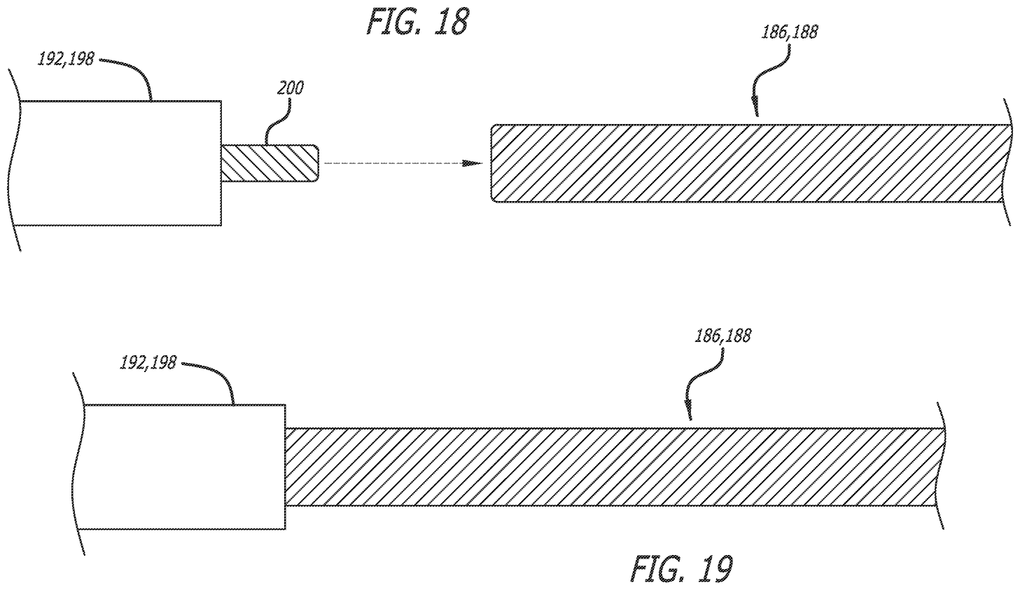

FIG. 15 illustrates inserting a proximal portion of the intraosseous needle 188 into the needle hub 192 in accordance with some embodiments, whereas FIGS. 16 and 17 illustrate the proximal portion of the intraosseous needle 188 within the needle hub 192 in accordance with some embodiments. FIG. 18 illustrates inserting the needle hub 192 into the proximal portion of the intraosseous needle 188 in accordance with some embodiments, whereas FIGS. 19 and 20 illustrate the needle hub 192 within the proximal portion of the intraosseous needle 188 in accordance with some embodiments.

When the intraosseous needle 188 has the one-piece shaft including the two longitudinal conductors 110, the intraosseous needle 188 operably connects to the intraosseous drill 190 when the intraosseous needle 188 is inserted into the needle hub 192 and the two longitudinal conductors 110 establish electrical connections with two electrical contacts 200 within the needle hub 192. (See FIG. 14 in view of FIG. 17 or 20.) In turn, the two electrical contacts 200 within the needle hub 192 directly or indirectly establish electrical connections with other electrical contacts including two brushes 202 in the intraosseous drill 190. Notably, the two longitudinal conductors 110 of the intraosseous needle 188 of FIG. 17 respectively include two contact points for establishing the electrical connections, which two contact points are on the abluminal side of the shaft 186 for establishing the electrical connections with the two electrical contacts 200 within the needle hub 192, which can be on two contact arms in the needle hub 192, as shown. Notably, the two longitudinal conductors 110 of the intraosseous needle 188 of FIG. 20 respectively include two contact points for establishing electrical connections, which two contact points are apportioned between the luminal side of the shaft 186 and an abluminal side of the shaft 186 for establishing the electrical connections with the two electrical contacts 200 within the needle hub 192, which can be a plug and a sleeve, as shown. In addition, due to the plug of the needle hub 192, an insulated obturator as set forth above can be disposed in a needle lumen of the intraosseous needle 188 and held in place therein by a pair of magnetic connectors between abutting ends of the plug and the obturator 196.

FIG. 21 illustrates the proximal portion of the intraosseous needle 188 within the needle hub 192 in accordance with some embodiments.

When the intraosseous needle 188 has the two-piece shaft 186 including the two longitudinal conductors 110, the intraosseous needle 188 operably connects to the intraosseous drill 190 when the intraosseous needle 188 is inserted into the needle hub 192 and the two longitudinal conductors 110 thereof establish an electrical connection with the electrical contact 200 within the needle hub 192, which can be on a contact arm in the needle hub 192, as shown. (See FIG. 14 in view of FIG. 21.) Likewise, the obturator 196 operably connects to the intraosseous drill 190 when the obturator 196 is inserted into the obturator hub 198 and the two longitudinal conductors 110 thereof establish an electrical connection with the electrical contact within the obturator hub 198. While not shown, the electrical contact within the obturator hub 198 can be on a contact arm in the obturator hub 198 similar to the contact arm in the needle hub 192. The electrical contact 200 within the needle hub 192 and the electrical contact within the obturator hub 198 directly or indirectly establish electrical connections with other electrical contacts including the two brushes 202 in the intraosseous drill 190.

Adverting to FIG. 13 to describe certain aspects of the intraosseous drill 190 as the impedance interrogator 102, the intraosseous drill 190, like the console 140 of the ultrasound system 126, includes the one-or-more processors and the primary memory including the ROM and the RAM. Such components can be incorporated into a microcontroller of the intraosseous drill 190. As set forth above, the instructions stored in the ROM are configured to instantiate one or more processes in the RAM for determining impedance from the electrical signals corresponding to the electrical currents passed through the biological materials or the non-biological materials. While the intraosseous drill 190 can include a display screen like the console 140 of the ultrasound system 126 for displaying information for a clinician during impedance interrogation with the intraosseous drill 190, the intraosseous drill 190 of FIG. 13 is shown with one or more visual status indicators, specifically one-or-more light-based indicators 204 including one-or-more light-emitting diodes (“LEDs”). Using, for example, the logic of the microcontroller, the one-or-more light-based indicators 204 can be configured to illuminate in accordance with biological of non-biological material between two tip electrodes formed by i) the two longitudinal conductors 110 at the tip of the intraosseous needle 188 or ii) the two longitudinal conductors 110 between tips of the intraosseous needle 188 and obturator 196. For example, a single light-based indicator can illuminate or change illumination colors when a medullary cavity of the patient P is accessed in accordance with the bone marrow therein. In another example, each light-based indicator of a number of the light-based indicators 204 sequentially illuminates when a corresponding tissue (e.g., soft tissue such as the epidermis, the dermis, or subcutaneous tissue; compact bone; or bone marrow) of the patient P is between the two tip electrodes. One or more aural status indicators can likewise be used; however, any sound-based indicators thereof should be substantially differentiated from sounds associated with operating the intraosseous drill 190.

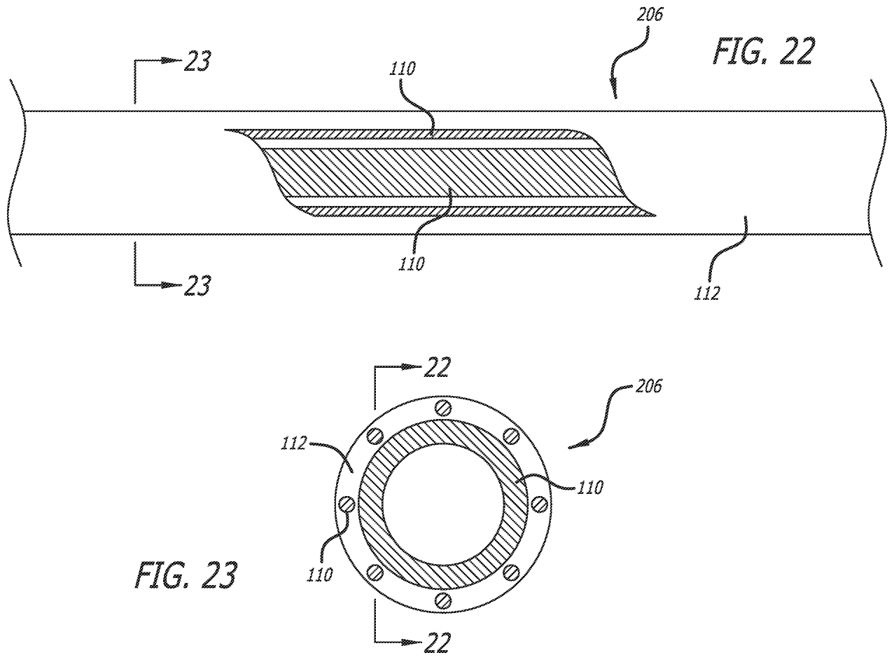

FIG. 22 illustrates the two-or-more longitudinal conductors 110 of a portion of the impedance-sensing medical device 104 in accordance with some embodiments. FIG. 23 illustrates a cross section of the portion of the impedance-sensing medical device 104 of FIG. 22 in accordance with some embodiments.

As shown, the impedance-sensing medical device 104 can include the two-or-more longitudinal conductors 110 in a two-piece shaft 206 with a single longitudinal conductor 110 in one piece of two pieces of the impedance-sensing medical device 104 and one or more longitudinal conductors 110 distributed among another piece of the two pieces of the impedance-sensing medical device 104. Such a configuration of the shaft 206 can include an inner tube of the single longitudinal conductor 110 and an outer tube including the one-or-more longitudinal conductors 110, wherein all longitudinal conductors 110 of the single longitudinal conductor 110 and the one-or-more longitudinal conductors 110 are separated from each other by the longitudinal insulator 112 of the outer tube. Alternatively, the outer tube is of the single longitudinal conductor 110 and the inner tube includes the one-or-more longitudinal conductors 110, wherein all longitudinal conductors 110 of the single longitudinal conductor 110 and the one-or-more longitudinal conductors 110 are separated from each other by the longitudinal insulator 112 of the inner tube.

Notwithstanding the foregoing, the two-piece shaft 206 can include an additional longitudinal insulator 112 over a luminal surface of the shaft 206, optionally with an entirety of the shaft 206 covered if surface portions of the shaft 206 are not configured for making electrical connections. Indeed, in an example, the two-piece shaft 206 can include the additional longitudinal insulator 112 over the luminal surface of the two-piece shaft 206 such that the additional longitudinal insulator is over the luminal surface of the inner tube of the shaft 206 shown in FIG. 23.

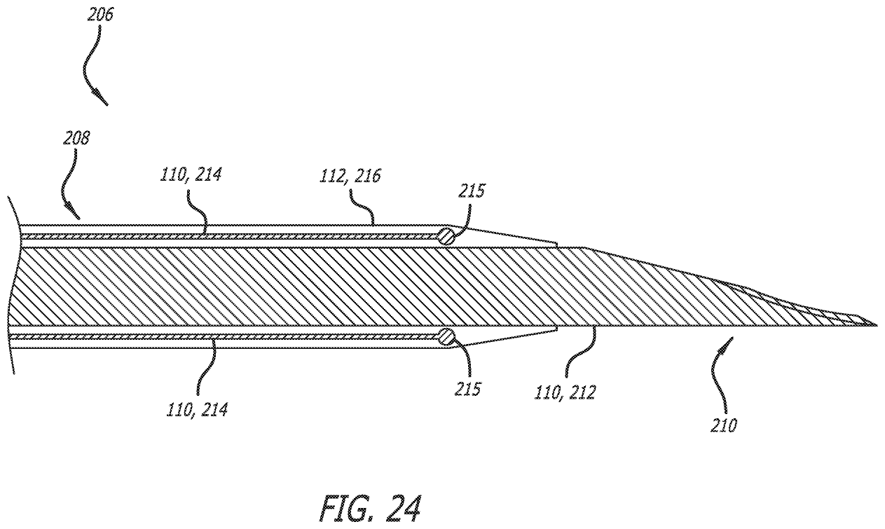

As set forth below with respect to FIG. 24, the one-or-more longitudinal conductors 110 distributed among the other piece of the two pieces of the impedance-sensing medical device 104 can be the one-or-more conductive filaments 214 disposed in the wall or septum of the impedance-sensing medical device 104, which one-or-more conductive filaments 214 terminate in the one-or-more electrodes 215. Such an impedance-sensing medical device 104 can be extruded with the one-or-more conductive filaments 214, or the one-or-more conductive filaments 214 can be embedded, optionally, as a flexible circuit on another flexible substrate, in at least the wall of the impedance-sensing medical device 104 after extrusion.

Alternatively, the one-or-more conductive filaments 214 can be lithographically patterned on at least the wall of the impedance-sensing medical device 104. Whether extruded or embedded, the one-or-more conductive filaments 214 can be exposed, optionally, in a subsequent skiving step to expose the one-or-more conductive filaments 214.

FIG. 24 illustrates the impedance-sensing medical device 104 of FIGS. 22 and 23 configured as a catheter 208 and a needle 210 disposed in the catheter 208 in accordance with some embodiments.

The impedance-sensing medical device 104 including the foregoing shaft 206 can be a combination of the catheter 208 and the needle 210 disposed in the catheter 208 configured for at least vascular access. In such a configuration, the two-or-more longitudinal conductors 110 can include the single longitudinal conductor 110 of a needle shaft 212 of the needle 210 and the one-or-more longitudinal conductors 110 as one-or-more conductive filaments 214 disposed in a catheter wall of a catheter tube 216 of the catheter 208, as shown, or a septum dividing the catheter tube 216 into two or more lumens. The one-or-more conductive filaments 214 terminate in one or more electrodes 215. The single longitudinal connector 110 of the needle shaft 212 and the one-or-more conductive filaments 214 are separated by the catheter wall or septum as the longitudinal insulator 112.

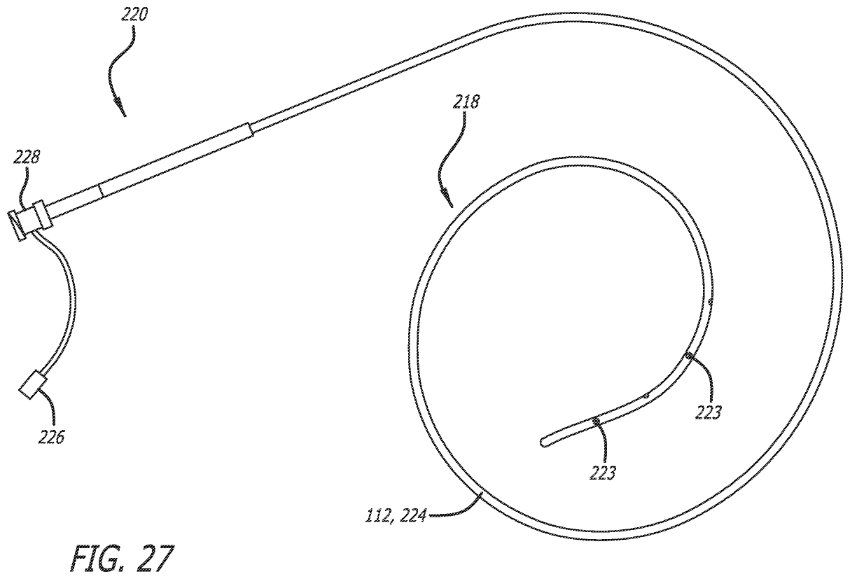

While not shown, the impedance-sensing medical device 104 of FIGS. 22 and 23 can be connected to the impedance interrogator 102 through a connector like that shown in FIG. 27, wherein the connector includes, for example, two terminals. A first terminal of the two terminals is indirectly electrically connected to the needle shaft 212 of the needle 210 when the needle 210 is disposed in the catheter 208 by way of an annular electrical contact around a luminal side of the catheter tube 216, a luminal side of a catheter hub over the catheter tube 216, or the like. A second pole of the two terminals is indirectly or directly electrically connected to the one-or-more conductive filaments 214 proximally extending from the catheter wall or the septum and, optionally, into a hub wall of the catheter hub or a suture wing extending therefrom.

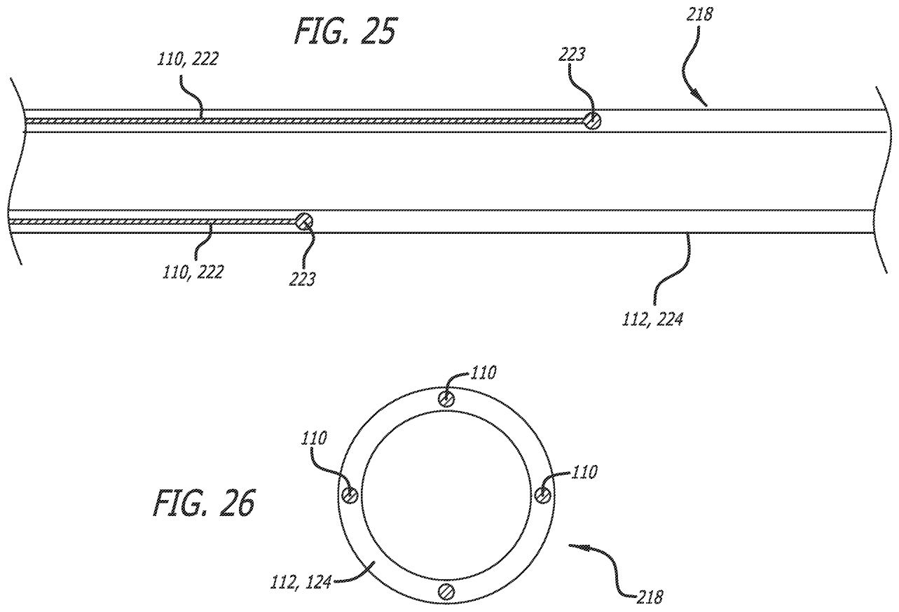

FIG. 25 illustrates the longitudinal conductors 110 of a portion of the impedance-sensing medical device 104 in accordance with some embodiments. FIG. 26 illustrates a cross section of the portion of the impedance-sensing medical device 104 of FIG. 25 in accordance with some embodiments.

As shown, the impedance-sensing medical device 104 can include the two-or-more longitudinal conductors 110 in a one-piece but optionally concentrically multilayered shaft 218 with the two-or-more longitudinal conductors 110 distributed among the one piece of the impedance-sensing medical device 104. Such a configuration of the shaft 218 can include a tube including the two-or-more longitudinal conductors 110, wherein all longitudinal conductors 110 of the two-or-more longitudinal conductors 110 are separated from each other by the longitudinal insulator 112 of the tube. Again, the shaft 218 can be multilayered, optionally with the two-or-more longitudinal conductors 110 separated from each other by concentric layers of the multilayered shaft 218, which concentric layers can be formed by coating (e.g., dip coating). Notably, as set forth below, the two-or-more longitudinal conductors 110 can include the two-or-more conductive filaments 222 that terminate in two or more electrodes 223. Such electrodes can be along a length of the impedance-sensing medical device 104 up to and including a distal tip of the impedance-sensing medical device 104.

Notwithstanding the foregoing, the one-piece shaft 218 can include an additional longitudinal insulator 112 over a luminal surface or abluminal surface of the shaft 218, optionally with an entirety of the shaft 218 covered if skived portions of the shaft 218 are not configured for making electrical connections.

Like that set forth above with respect to FIGS. 22 and 23, the one-or-more longitudinal conductors 110 distributed about the shaft 218 of the impedance-sensing medical device 104 can be the two-or-more conductive filaments 222 disposed in a wall of the shaft 218, which two-or-more conductive filaments 222 terminate in the two-or-more electrodes 223. Such an impedance-sensing medical device 104 can be extruded with the two-or-more conductive filaments 222, or the two-or-more conductive filaments 214 can be embedded, optionally, as a flexible circuit on another flexible substrate, in at least the wall of the shaft 218 after extrusion. Alternatively, the two-or-more conductive filaments 222 can be lithographically patterned on at least the wall of the shaft 218. Whether extruded or embedded, the two-or-more conductive filaments 222 can be exposed, optionally, in a subsequent skiving step to expose the two-or-more conductive filaments 222.

FIG. 27 illustrates the impedance-sensing medical device 104 of FIGS. 25 and 26 configured as a catheter 220 in accordance with some embodiments.

The impedance-sensing medical device 104 including the foregoing shaft 218 can be the catheter 220 configured for at least vascular access. In such a configuration, the two-or-more longitudinal conductors 110 include two-or-more conductive filaments 222 disposed in a catheter wall of a catheter tube 224 of the catheter 220 or a septum dividing the catheter tube 224 into two or more lumens, which two-or-more conductive filaments 222 terminate in the two-or-more electrodes 223 along a length of the catheter 220 up to and including a catheter tip of the catheter 220. The two-or-more conductive filaments 222 are separated by the catheter wall or septum as the longitudinal insulator 112. Such a catheter as the impedance-sensing medical device 104 is useful in detecting air bubbles or occlusions in the catheter 220. Such a catheter is even capable of electrocardiography (“ECG”) when fitted with an ECG stylet.

The impedance-sensing medical device 104 can be connected to the impedance interrogator 102 through a connector 226 extending from a catheter hub 228, a suture wing extending from the catheter hub 228, a Luer connector of an extension leg, or the like. Indeed, even saline columns through the two-or-more lumens, when present, can be used to connect the impedance-sensing medical device 104 to the impedance interrogator 102. While not shown, the connector 226 includes, for example, two or more terminals. At least a first terminal of the two terminals is indirectly or directly electrically connected to a first conductive filament of the two-or-more conductive filaments 222 proximally extending from the catheter wall or the septum and, optionally, into a hub wall of the catheter hub or the suture wing extending therefrom. Likewise, at least a second terminal of the two terminals is indirectly or directly electrically connected to a second conductive filament of the two-or-more conductive filaments 222 proximally extending from the catheter wall and, optionally, into the hub wall of the catheter hub or the suture wing.

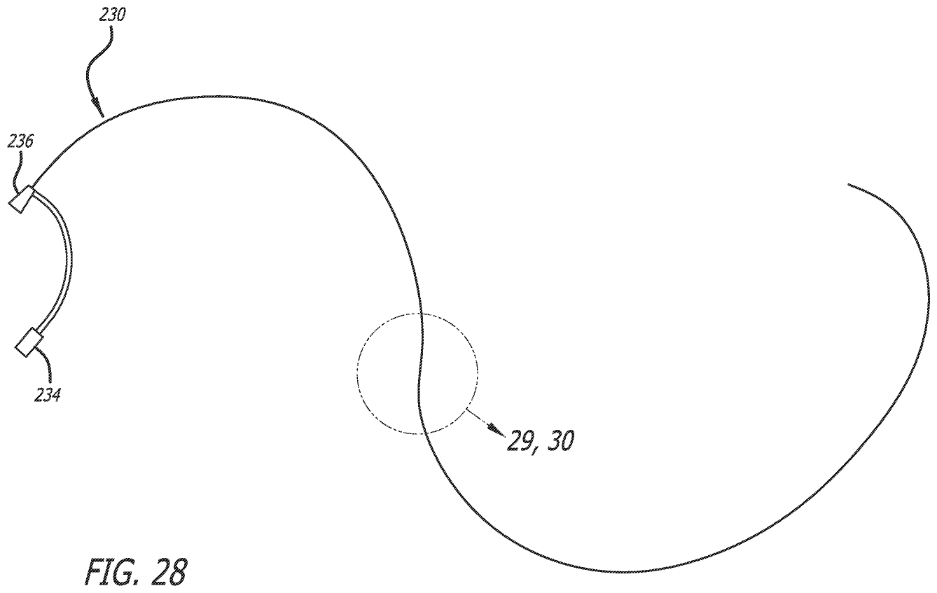

FIGS. 28-30 illustrate the impedance-sensing medical device 104 configured as a guidewire 230 in accordance with some embodiments.

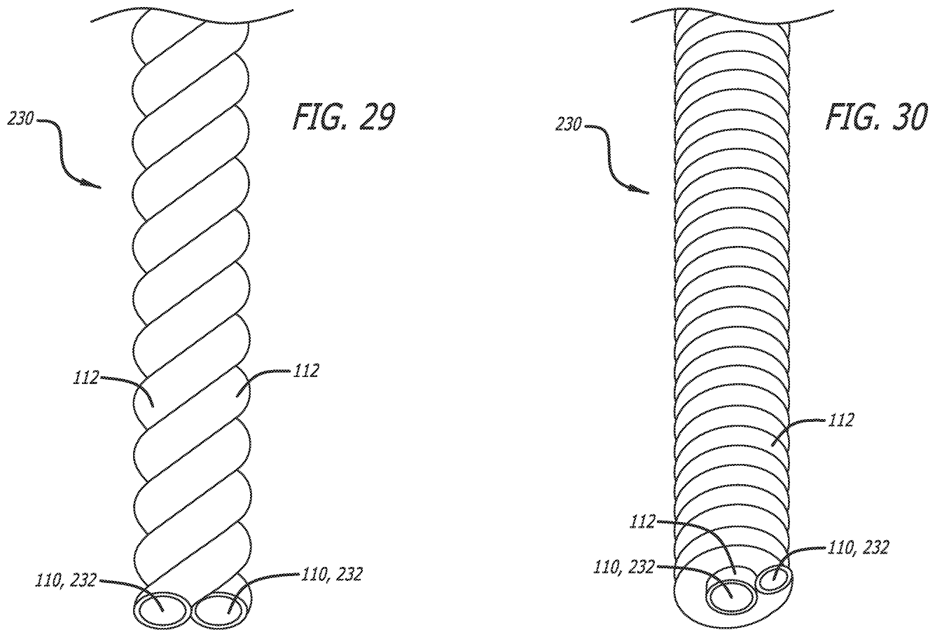

As shown, the impedance-sensing medical device 104 can include the two-or-more longitudinal conductors 110 distributed in one piece of the impedance-sensing medical device 104 such as the guidewire 230, which is configured for at least vascular access or vascular guidance. The two-or-more longitudinal conductors 110 include a twisted pair of conductive filaments 232 as shown in FIG. 29. At least one conductive filament of the twisted pair of conductive filaments 232 is coated with a polymeric material as the longitudinal insulator 112 of one-or-more longitudinal insulators 112. Alternatively, the two-or-more longitudinal conductors 110 include a wound pair of the conductive filaments 232 with a wound conductive filament wound around a core conductive filament as shown in FIG. 30. At least one conductive filament of the wound and core conductive filaments 232 is coated with a polymeric material as the longitudinal insulator 112 of the one-or-more longitudinal insulators 112.

The impedance-sensing medical device 104 can be connected to the impedance interrogator 102 through a connector 234 extending from a guidewire hub 236, wherein the connector 234 includes, for example, two or more terminals. While not shown in detail, at least a first terminal of the two terminals is indirectly or directly electrically connected to a first conductive filament of the two-or-more conductive filaments 232 in a proximal portion of the guidewire 230. Likewise, at least a second terminal of the two terminals is indirectly or directly electrically connected to a second conductive filament of the two-or-more conductive filaments 232 in the proximal portion of the guidewire 230.

Any connection between the impedance-sensing medical device 104 and the impedance interrogator 102 set forth above can be made a magnetic connection with complementary magnetic connectors including, for example, complementary magnets (e.g., neodymium magnets).

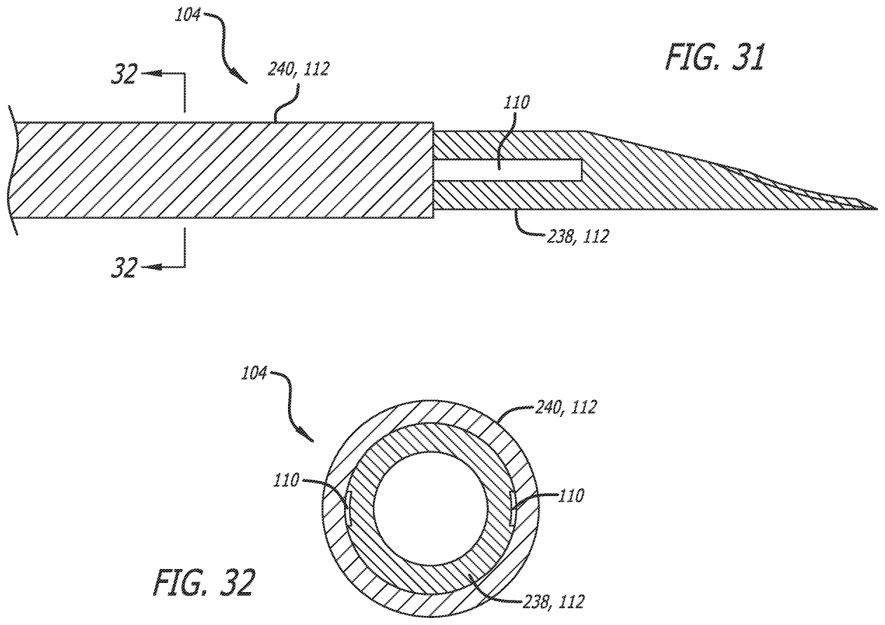

FIG. 31 illustrates the impedance-sensing medical device 104 configured as a needle 238 with an outer tube 240 slidably disposed thereover in accordance with some embodiments. FIG. 32 illustrates a cross section of a portion of the impedance-sensing medical device 104 of FIG. 31 in accordance with some embodiments.

As shown, the impedance-sensing medical device 104 includes, in some embodiments, a two-piece shaft including an inner tube configured as the needle 238 and the outer tube 240 slidably disposed thereover. Two or more longitudinal conductors 110 can be distributed among the inner tube (or needle 238) and the outer tube 240 of the two-piece shaft with a longitudinal insulator 112 of the inner tube (or needle 238) or the outer tube 240 separating the two-or-more longitudinal conductors 110 from each other. The inner tube (or needle 238) of the two-piece shaft can be formed of the longitudinal insulator 112 or coated with the longitudinal insulator 112, and the outer tube 240 of the two-piece shaft can be formed of a same or different longitudinal insulator 112 as the inner tube (or needle 238) of the two-piece shaft, thereby separating the two-or-more longitudinal conductors 110 from each other.

The two-or-more longitudinal conductors 110 can be distributed over the inner tube (or needle 238) of the two-piece shaft such as by embedding or lithographically patterning the two-or-more longitudinal conductors 110 over the inner tube (or needle 238) of the two-piece shaft. For example, the needle 238 can be stainless steel, the needle 238 can be coated with the longitudinal insulator 112, and the two-or-more longitudinal conductors can be lithographically patterned in a same or different longitudinal insulator over that coating the needle. And like that set forth above, the two-or-more conductors 110 are configured to emit, detect, or alternately emit and detect via two or more electrodes thereof electrical currents passed through a biological or non-biological material. While not shown, the impedance-sensing medical device 104 is configured, for example, with a connector (e.g., the connector 226 of FIG. 27) such as a multiple-pin electrical header to form a direct or indirect connection to the impedance interrogator 102 and provide electrical signals to the impedance interrogator 102. Indeed, the connector includes two or more connection points (e.g., two or more pins in the multiple-pin electrical header) in correspondence with the two-or-more electrodes of the two-or-more longitudinal conductors 110.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

1-8. (canceled)

9. An impedance-determining medical system, comprising:
an impedance interrogator including one or more processors, primary memory including read-only memory (“ROM”) and random-access memory (“RAM”), and instructions stored in the ROM configured to instantiate one or more processes in the RAM for determining impedance from electrical signals corresponding to electrical currents passed through a biological or non-biological material; and
an impedance-sensing medical device including:
two or more longitudinal conductors distributed among one or more pieces of the impedance-sensing medical device and separated by one or more longitudinal insulators, the two-or-more conductors configured to emit, detect, or alternately emit and detect via two or more electrodes thereof the electrical currents passed through the biological or non-biological, and the impedance-sensing medical device configured to form a direct or indirect connection to the impedance interrogator and provide the electrical signals to the impedance interrogator.

10. The impedance-determining medical system of claim 9, wherein the impedance-sensing medical device includes the two-or-more longitudinal conductors in a one-piece shaft with two longitudinal conductors separated by two longitudinal insulators.

11. The impedance-determining medical system of claim 10, wherein each longitudinal conductor of the two longitudinal conductors forms less than half the shaft, each longitudinal insulator of the two longitudinal insulators disposed between the two longitudinal conductors on an opposite side of the shaft from another longitudinal insulator of the two longitudinal insulators.

12. The impedance-determining medical system of claim 10, wherein the shaft has a symmetry such that a longitudinal plane of symmetry passes through either the two longitudinal conductors or the two longitudinal insulators but not both the two longitudinal conductors and the two longitudinal insulators.

13. The impedance-determining medical system of claim 10, wherein the impedance-sensing medical device is a needle configured for at least vascular access.

14. The impedance-determining medical system of claim 13, wherein the needle includes a needle hub over a proximal portion of the shaft and a needle tip formed in a distal portion of the shaft.

15. The impedance-determining medical system of claim 13, further comprising a needle guide configured to couple with a needle-guide attachment point of an ultrasound probe as the impedance interrogator or a portion thereof, the needle guide, the needle-guide attachment point, and the ultrasound probe including electrical circuitry configured to operably connect the needle to the ultrasound probe when a) the needle guide is coupled with the needle-guide attachment point and b) the needle is inserted into an aperture of the needle guide such that the two longitudinal conductors make electrical connections with two opposing electrical contacts within the aperture.

16. The impedance-determining medical system of claim 15, wherein the needle guide includes conductive inward-facing protrusions configured to establish an electrical connection within outward-facing receptacles of the needle-guide attachment point when the needle guide is coupled with the needle-guide attachment point.

17. The impedance-determining medical system of claim 16, wherein the protrusions include barrier-piercing contact points configured to a) pierce a protective film-based barrier when used over the ultrasound probe and b) establish the electrical connection with contact points within the receptacles of the needle-guide attachment point, the receptacles being shaped to accommodate the barrier-piercing contact points of the protrusions.

18. The impedance-determining medical system of claim 10, further comprising an alligator-clamp connecting device including a proximal connecting-device connector configured to couple with an impedance-interrogator connector of the impedance interrogator, a distal connecting-device connector including an alligator clamp configured to couple with the impedance-sensing medical device, and electrical circuitry therebetween.

19. The impedance-determining medical system of claim 18, wherein the alligator clamp includes a pair of jaws, each jaw of the pair of jaws including a plurality of teeth configured to establish an electrical connection with a longitudinal conductor of the two longitudinal conductors when the alligator clamp is clamped over the shaft.

20. The impedance-determining medical system of claim 19, wherein the teeth include barrier-piercing contact points configured to pierce a protective film-based barrier when used over a portion of the shaft.

21. The impedance-determining medical system of claim 9, wherein the impedance-sensing medical device includes the two-or-more longitudinal conductors in a one-piece shaft with two longitudinal conductors separated by one longitudinal insulator.

22. The impedance-determining medical system of claim 21, wherein each longitudinal conductor of the two longitudinal conductors is primarily disposed on an opposite side of the shaft from another longitudinal conductor of the two longitudinal conductors, sides of the shaft being a luminal side of the shaft and an abluminal side of the shaft.

23. The impedance-determining medical system of claim 22, wherein the two longitudinal conductors respectively include two contact points for establishing electrical connections, the two contact points on the abluminal side of the shaft.

24. The impedance-determining medical system of claim 22, wherein the two longitudinal conductors respectively include two contact points for establishing electrical connections, the two contact points apportioned between the luminal side of the shaft and an abluminal side of the shaft.

25. The impedance-determining medical system of claim 23, wherein the impedance-sensing medical device is a needle configured for at least intraosseous access.

26. The impedance-determining medical system of claim 25, wherein the impedance interrogator is an intraosseous drill including a needle hub configured to hold a proximal portion of the shaft and operably connect the needle to the intraosseous drill when the needle is inserted into the needle hub and the two longitudinal conductors establish the electrical connections with two electrical contacts within the needle hub.

27. The impedance-determining medical system of claim 26, wherein the intraosseous drill includes an obturator hub over the needle hub, the obturator hub configured to hold a proximal portion of an insulated obturator or an obturator of an insulator material.

28. The impedance-determining medical system of claim 9, wherein the impedance-sensing medical device includes the two-or-more longitudinal conductors in a two-piece shaft with two longitudinal conductors separated by one longitudinal insulator.

29. The impedance-determining medical system of claim 28, wherein the impedance-sensing medical device includes a combination of a needle and an obturator disposed in the needle configured for at least intraosseous access.

30. The impedance-determining medical system of claim 29, wherein the longitudinal insulator is disposed over a luminal side of the needle or an abluminal side of the obturator.

31. The impedance-determining medical system of claim 29, wherein the impedance interrogator is an intraosseous drill including a needle hub configured to hold a proximal portion of the needle and operably connect the needle to the intraosseous drill when the needle is inserted into the needle hub and a longitudinal conductor of the needle establishes an electrical connection with an electrical contact within the needle hub.

32. The impedance-determining medical system of claim 31, wherein the intraosseous drill includes an obturator hub over the needle hub, the obturator hub configured to hold a proximal portion of the obturator and operably connect the obturator to the intraosseous drill when the obturator is inserted into the obturator hub and a longitudinal conductor of the obturator establishes an electrical connection with an electrical contact within the obturator hub.

33. The impedance-determining medical system of claim 9, wherein the impedance-sensing medical device includes the two-or-more longitudinal conductors distributed among two pieces of the impedance-sensing medical device including a catheter and a needle disposed in the catheter configured for at least vascular access.

34. The impedance-determining medical system of claim 33, wherein the two-or-more conductors include a needle shaft of the needle and one or more conductive filaments disposed in a catheter wall of a catheter tube of the catheter, the needle shaft and the one-or-more conductive filaments separated by the catheter wall as the insulator of the one-or-more insulators.

35. The impedance-determining medical system of claim 9, wherein the impedance-sensing medical device includes the two-or-more longitudinal conductors distributed in one piece of the impedance-sensing medical device including a catheter configured for at least vascular access.

36. The impedance-determining medical system of claim 35, wherein the two-or-more conductors include two-or-more conductive filaments disposed in a catheter wall of the catheter tube of the catheter, the two-or-more conductive filaments separated by the catheter wall as the insulator of the one-or-more insulators.

37. The impedance-determining medical system of claim 9, wherein the impedance-sensing medical device includes the two-or-more longitudinal conductors distributed in one piece of the impedance-sensing medical device including a guidewire configured for at least vascular access or vascular guidance.

38. The impedance-determining medical system of claim 37, wherein the two-or-more conductors include a twisted pair of conductive filaments, at least one conductive filament of the conductive filaments coated with a polymeric material as the insulator of the one-or-more insulators.

39. The impedance-determining medical system of claim 37, wherein the two-or-more conductors include a wound pair of conductive filaments with a wound conductive filament wound around a core conductive filament, at least one conductive filament of the conductive filaments coated with a polymeric material as the insulator of the one-or-more insulators.
